(19) United States
(12) Patent Application Publication
(10) Pub. No.: US 2007/0260214 A1
(43) Pub. Date: Nov. 8, 2007

(54) Medical procedure through natural body opening

(75) Inventors: Takayasu Mikkaichi, Tokyo (JP); Tetsuya Yamamoto, Hanno-shi (JP); Kunihide Kaji, Tokyo (JP); Kensei Nakahashi, Tokyo (JP)

Correspondence Address: SCULLY SCOTT MURPHY & PRESSER, PC, 400 GARDEN CITY PLAZA, SUITE 300, GARDEN CITY, NY, 11530, US

(73) Assignee: OLYMPUS MEDICAL SYSTEMS CORP., TOKYO (JP)

(21) Appl. No.: 11/331,967

(22) Filed: Jan. 13, 2006

(51) Int. Cl. A61M 31/00

(52) U.S. Cl. 604/500

(57) ABSTRACT

A medical procedure through a natural body orifice according to the present invention includes: introducing a first device having an insertion portion, from a patient's natural orifice into a hollow organ; performing a first medical procedure using the first device; withdrawing the first device from the body, and introducing a second device having an insertion portion, from a patient's natural orifice into the body; and performing a second medical procedure using the second device, wherein the first medical procedure is performed prior to the second medical procedure for performing the second medical procedure.

### BACKGROUND OF THE INVENTION

1. Field of the Invention

The present invention relates to a medical procedure or action through a natural body orifice.

2. Description of Related Art

In the case where a procedure is performed on a human organ and the like, there is known a laparoscopic operation for manipulating by opening a plurality of orifices in the abdominal wall, instead of largely incising the abdominal wall, and then inserting treatment tools such as a rigid laparoscope and a forceps into the respective orifices. Since it can be completed simply by opening small orifices, there are advantages of less invasion and quick recovery of the patient.

Moreover, recently, as a method of further reducing invasion of a patient, there is proposed a procedure by inserting an endoscope from a natural orifice such as the patient's mouth, a nostril, and the anus. An example of such a medical procedure is disclosed in U.S. Pat. No. 5,458,131. A flexible endoscope is inserted from the mouth of a patient receiving insufflation treatment, and the endoscope is introduced from an orifice formed in the stomach wall into the abdominal cavity. The endoscope is used for monitoring inside the abdominal cavity. Furthermore, an organ is treated by using a treatment tool passed through the endoscope, and a treatment tool inserted from another orifice opened in the stomach, or an orifice opened in the sigmoid colon from the anus. After the procedure in the abdominal cavity is completed, the treatment tools are withdrawn, and the orifices are closed. Upon closure of an orifice, the tissue around the orifice is drawn together into a bundle, and then the tissue is tied with an O-ring.

### SUMMARY OF THE INVENTION

A medical procedure through a natural body orifice according to a first aspect of the present invention comprises: introducing a first device having an insertion portion, from a patient's natural orifice into a hollow organ; performing a first medical procedure using the first device; withdrawing the first device from the body, and introducing a second device having an insertion portion, from a patient's natural orifice into the body; and performing a second medical procedure using the second device, wherein the first medical procedure is performed prior to the second medical procedure for performing the second medical procedure.

A medical procedure through a natural body orifice according to a second aspect of the present invention comprises: introducing a first device having an insertion portion, from a patient's natural orifice into the body; introducing a flexible guide member from the patient's natural orifice into the body; performing a first medical procedure using the first device; activating a shape retention function of the guide member from outside of the body, so as to fix a curved shape of the guide member according to a curved shape of the insertion portion of the first device; withdrawing the first device from the body, introducing a second device having an insertion portion, from a patient's natural orifice into the body, along the fixed shape of the guide member; and performing a second medical procedure using the second device, wherein the first medical procedure is performed prior to the second medical procedure for performing the second medical procedure.

### BRIEF DESCRIPTION OF THE DRAWINGS

FIG. 1 is an explanatory diagram of a medical procedure, showing a device inserted into a stomach.

FIG. 2A is a view showing a forward view type endoscope.

FIG. 2B is an enlarged view of the distal part of FIG. 2A.

FIG. 3A is a view showing a lateral view type endoscope.

FIG. 3B is an enlarged view of the distal part of the endoscope of FIG. 3A.

FIG. 4 is an enlarged cross-sectional view of the distal part of an oblique view type endoscope.

FIG. 5 is a view showing a construction of a treatment tool operation stage.

FIG. 6 is a view on the arrow A of FIG. 4, for explaining the operation of the treatment tool operation stage.

FIG. 7A is a view showing one aspect of a construction of an endoscope having an ultrasonic diagnostic function, for which the distal end is shown in an enlarged cross-sectional view.

FIG. 7B is the enlarged view of the distal end of the endoscope of FIG. 7A.

FIG. 8 is a view showing one aspect of a construction of an endoscope having a narrow-band optical observation function.

FIG. 9 is a view showing an orifice formed in the stomach wall.

FIG. 10 is a view showing a device introduced from the orifice of the stomach into the abdominal cavity.

FIG. 11 is a view showing an endoscope having two bending sections, as one aspect of a multistep bendable endoscope.

FIG. 12 is an explanatory diagram of an operation of making the distal face of the endoscope approach to a target site by means of two-step bending.

FIG. 13 is a view showing one aspect of a construction of an endoscope having a fluorescent observation function.

FIG. 14 is a view showing one aspect of an observation device capable of changing the view field angle.

FIG. 15 is a view showing one aspect of a construction of an endoscope having two optical systems.

FIG. 16 is a view showing the distal end of the endoscope having the two optical systems of FIG. 15.

FIG. 17 is a view showing one aspect of a treatment endoscope.

FIG. 18 is a view showing an example of a video image that can be obtained by the observation device of the system shown in FIG. 17.

FIG. 19 is an explanatory diagram of one aspect of a medical procedure when the treatment endoscope is used.

FIG. 20 is an explanatory diagram of a medical procedure, showing a device inserted into a stomach.

FIG. 21 is a view showing an orifice formed in the stomach wall.

FIG. 22 is a cross-sectional view showing one aspect of an overtube having a shape retention function.

FIG. 23 is a view showing a device introduced from the orifice of the stomach into the abdominal cavity.

FIG. 24 is a view showing one aspect of suturing the orifice in the stomach wall after the medical procedure is completed in the abdominal cavity.

### DETAILED DESCRIPTION OF THE INVENTION

Hereunder is a detailed description of a medical procedure of the embodiment. The following is a description of a medical procedure as an example, in which an endoscope is inserted from a patient's mouth, and an orifice is formed in the stomach wall to access to the abdominal cavity, so as to observe or treat a tissue (a target site) serving as a part of an organ in the abdominal cavity. However, the natural orifice to be inserted with a medical device is not limited to the mouth, and may be any orifice as long as it is a natural body orifice such as a nostril and the anus. Moreover, the contents of the medical procedure are applicable to various procedures such as suture, observation, incision, and cell sampling. More specifically, medical procedures such as observation of an organ in the abdominal cavity, biopsy of the liver or pancreas, cautery of the uterus myoma or liver (and other organs), sterilization treatment, extirpation of the appendix or gallbladder, and operations for obesity, are performed. The same reference symbols are used for the same components, and duplicate description is omitted.

### First Embodiment

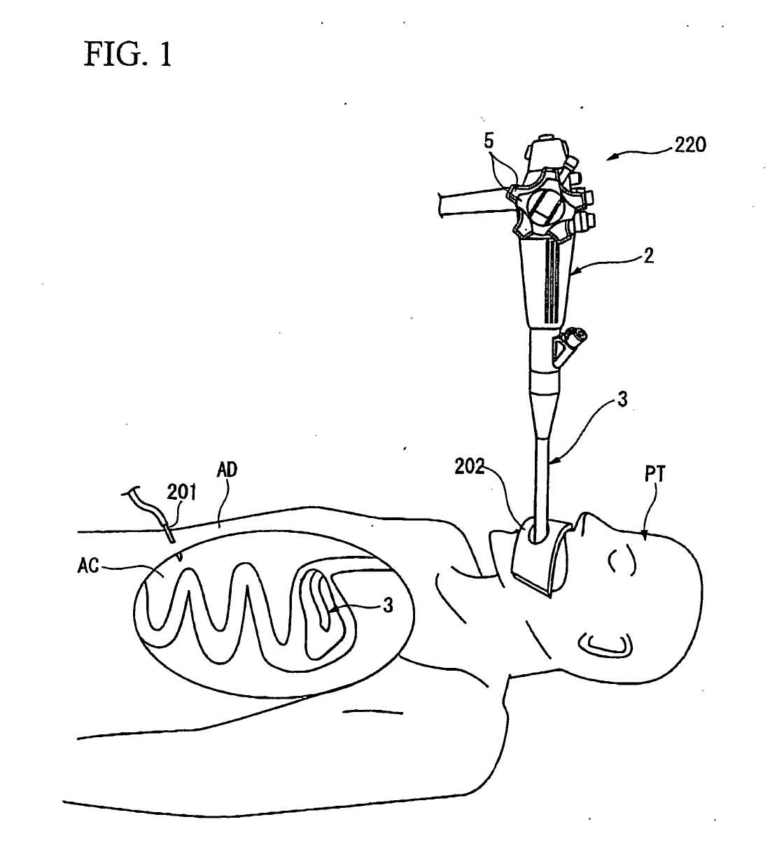

Firstly, as shown in FIG. 1, an insertion portion of a device (first device) for performing a medical procedure on a living body, is inserted into a hollow organ through a natural orifice.

A patient PT is laid on his back so that the abdomen AD is faced upwards. Then an endoscope (first device 220 for performing a medical procedure) is inserted from a mouthpiece 202 attached to the patient PT's mouth, into the stomach ST.

Considering the view field direction of an observation device, the endoscope serving as the first device 220 can be largely classified into a forward view type having a view field toward the insertion direction of the endoscope, a lateral view type having a view field in a direction approximately orthogonal to the insertion direction, and a oblique view type having a view field in a direction intersecting the insertion direction.

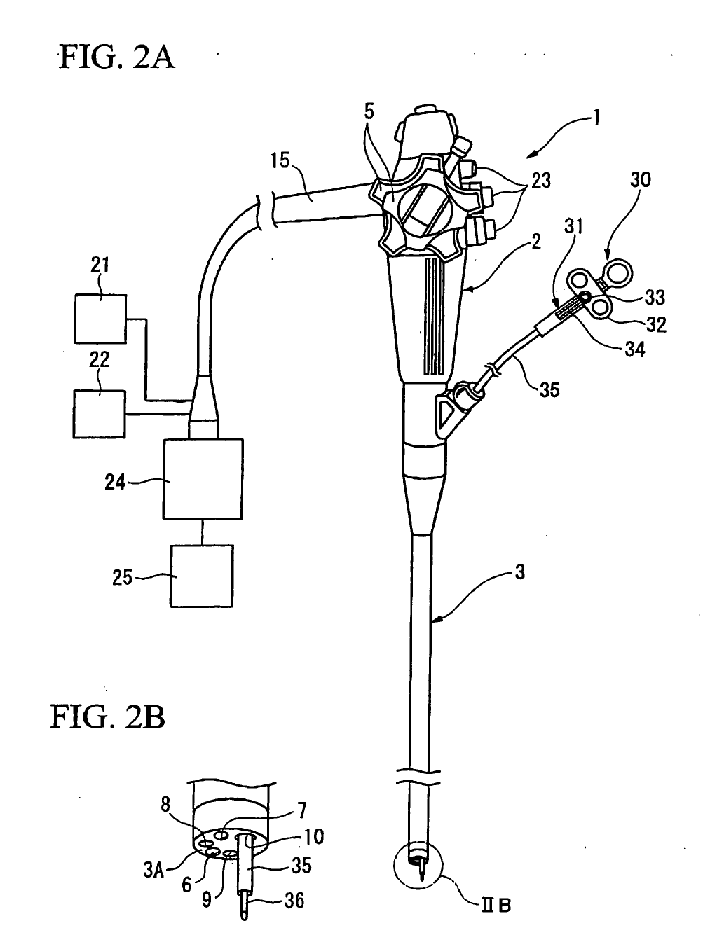

As shown in FIG. 2A and FIG. 2B, in a forward view type endoscope (forward view observation device) 1, an insertion portion 3 to be inserted into a patient's body, is extended from an operation portion 2 which is operated by an operator. The insertion portion 3 is slender and flexible. The distal end of the insertion portion 3 becomes a bending section 4 which can be curved by an angle knob 5 of the operation portion 2. The insertion portion 3 is provided with one bending section 4, which is constituted to be bendable in two directions or four directions (two up-and-down directions or four up-and-down and left-and-right directions). Moreover, there is provided one operation system (angle knob 5) that is operated in order to curve the bending section 4 in a desired condition. In this embodiment, the construction is such that the bending section 4 is bendable in four directions (four up-and-down and left-and-right directions with respect to an observation image), and the angle knob 5 has a first knob and a second knob for bending the bending section 4 in up-and-down directions with respect to the observation image. A distal face 3A of the insertion portion 3 is arranged with an illuminating device 7 and an observation device 6 for observing inside the body. The view field direction of the observation device 6 is set such that the distal face 3A is oriented forward (insertion direction) in the longitudinal direction of the insertion portion 3, which is suitable for obtaining an endoscopic image in the insertion direction. The observation device 6 comprises, for example, an observational optical system such as an object lens, and a CCD (Charged Coupled Device) as an imager. The observational optical system is arranged so that an optical axis and the insertion direction are in parallel. The CCD is arranged in an imaging point of the object lens in parallel with the object lens (if a beam emitting from the object lens is refracted by a prism, the photo receiving surface of the CCD can be arranged in a different direction other than the insertion direction). The illuminating device 7 has a construction where illuminating light is guided from a light source unit outside of the body, by an optical fiber, and the inside of the body is illuminated through an observation window.

Moreover, the distal face 3A of the insertion portion 3 is arranged with distal orifices of various channels 8 to 10. A fluid supply channel 8 is a duct used for supplying a fluid into the body. A suction channel 9 is a duct used for sucking a fluid from the body, or for sucking remnants (such as residues and filth) in the body. A work channel 10 is a work channel through which a treatment tool is passed. The respective channels 8 to 10 are extended from the insertion portion 3 towards the operation portion 2. The proximal orifice of the work channel 10 is provided on the side of the operation portion 2. The other channels 8 and 9 are connected to a fluid supply device 21 and a suction device 22 through a universal cable 15. The construction may be such that the functions of the suction channel 9 and the work channel 10 are performed by a common channel, or may be such that the functions of the fluid supply channel 8, the suction channel 9, and the work channel 10 are performed by a single channel. A fluid supply operation and a suction operation are performed by switches 23 arranged on the operation portion 2. Furthermore, the endoscope 1 is also connected to a controller 24, through the universal cable 15. The controller 24 is a device which controls the endoscope 1, and is installed with an image processor and a light source, being capable of outputting various images (video images) to a monitor 25.

Moreover, a high frequency knife 30 as an example of the treatment tool, is passed through the treatment tool channel 10 of the endoscope 1. The high frequency knife 30 is used for incising a tissue, or marking a tissue. An operation portion 31 operated by an operator is provided with a back-and-forth movable handle 32. The handle 32 is provided with a terminal 33, and is connectable to a high frequency power source (not shown). The terminal 33 is electrically connected to a transmission member 34 covered with a conductive wire. The transmission member 34 is led into a flexible insertion portion 35 extended from the operation portion 31. The insertion portion 35 is passed through the work channel 10, and projected from the distal face 3A of the insertion portion 3. The distal end of the high frequency knife 30 is provided with a treatment portion 36 in a projectable manner. If electricity is charged through the transmission member 34 while the treatment portion 36 is projected from the insertion portion 35, then a tissue can be incised or the like using the treatment portion 36, by an operation from outside the body.

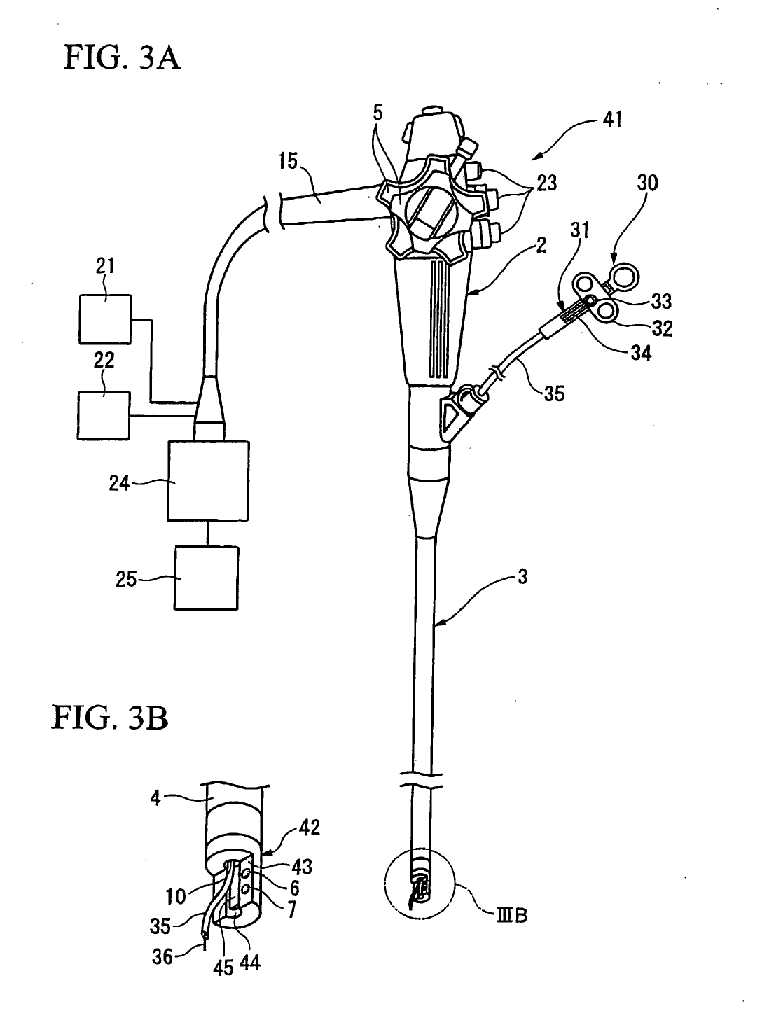

As shown in FIGS. 3A and 3B, a lateral view type endoscope (lateral view observation device) 41 is provided with a distal hard part 42 at the tip of the bending section 4 of the insertion portion 3. The distal hard part 42 has a notch 43 formed by cutting a column stage having the distal end formed in a spherical shape, by a plane offset from the central axis. In the notch 43, a channel orifice 44 is opened slenderly along the insertion direction. The channel orifice 44 is communicated with the work channel 10, and is provided with a treatment tool operation stage 45 inside thereof. The treatment tool operation stage 45 is used for raising the distal end of a treatment tool in a predetermined direction. The proximal end of the treatment tool operation stage 45 is supported on the distal hard part 42 by a pin (not shown). When the operation portion 2 of the endoscope 41 is operated, the distal end of the treatment tool operation stage 45 can be rotated using the pin as a fulcrum, so as to rise from the channel orifice 44. As a result, the orientation of the treatment tool can be changed without bending the insertion portion 3 of the endoscope 41. The constructions of the bending section 4 and the angle knob 5 are approximately the same as those of the abovementioned forward view observation device.

Furthermore, the notch 43 of the distal hard part 42 is arranged with the observation device 6 and the illuminating device 7 sequentially from the proximal side along the channel orifice 44. The observation device 6 is arranged with the observational optical system and the CCD in parallel with the insertion direction, and has a view field in a direction approximately orthogonal to the insertion direction of the insertion portion 3. In the distal end of the endoscope 41, a fluid supply channel and a suction channel (not shown) are opened. Furthermore, if the optical axis is curved using a prism, the CCD can be arranged orthogonally with respect to the insertion direction. The detailed construction of this endoscope 41 is disclosed in U.S. Pat. No. 6,827,683. The contents disclosed in U.S. Pat. No. 6,827,683 are incorporated in the present embodiment.

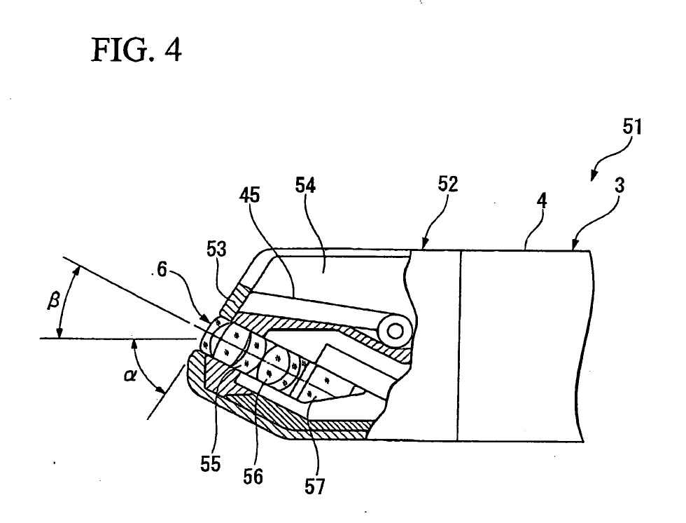

As shown in FIG. 4, an oblique view type endoscope (oblique view observation device) 51 is different from the endoscope 41 only in a distal hard part 52 provided at the tip of the bending section 4 of the insertion portion 3. The extremity of the distal hard part 52 is formed with an inclined plane 53 inclined at a predetermined angle α with respect to the insertion direction. This inclined plane 53 is arranged with the observation device 6 and the illuminating device 7 (not shown), and is formed with a channel orifice 54. The observation device 6 is arranged so that an object lens group 55 has an optical axis orthogonal to the inclined plane 53. The CCD 57 is arranged in the imaging position of a beam from the object lens group 55 through an imaging lens group 56. Therefore, this observation device 6 has a view field where the view field direction is shifted by an angle β with respect to the forward view type endoscope 1. The channel orifice 54 is provided to be inclined with respect to the insertion direction, and is communicated with the work channel 10. The channel orifice 54 is arranged with the treatment tool operation stage 45. This oblique view type endoscope 51 is characterized in that an area between ahead of the insertion direction and a direction having an angle with respect to the insertion direction can be captured in the view field. The constructions of the bending section 4 and the angle knob 5 are approximately the same as those of the abovementioned forward view observation device.

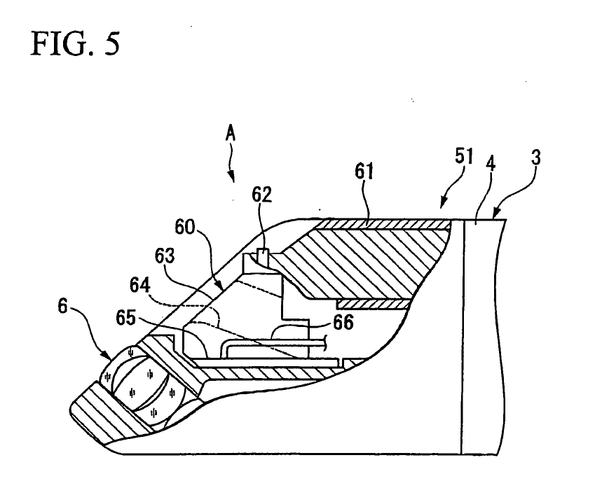

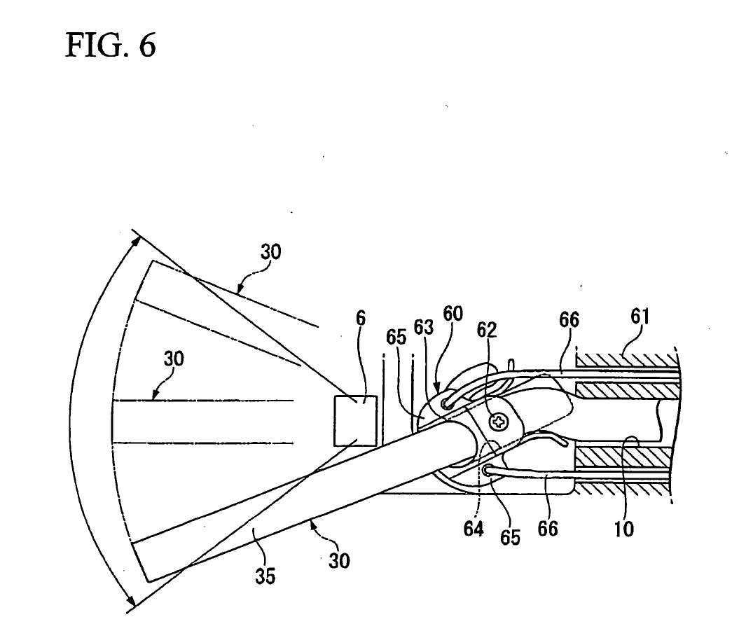

Here, the treatment tool operation stage 45 is not an essential construction in the endoscopes 41 and 51. Furthermore, together with the treatment tool operation stage 45, or instead of the treatment tool operation stage 45, there may be provided a treatment tool operation stage 60 as shown in FIG. 5 and FIG. 6. This treatment tool operation stage 60 swings a tip of a treatment tool in the left-and right direction (orthogonal direction to the page of FIG. 5, and up-and down direction on the page of FIG. 6). FIG. 5 and FIG. 6 show an example of applying it to the oblique view type endoscope 51, however it may be provided on the lateral view type endoscope 41.

The treatment tool operation stage 60 has a main body 63 swingably attached to a distal hard part 61, by a pin 62. The pin 62 is arranged so that the extension line of the work channel 10 and the extension line of the pin 62 intersect. The main body 63 is formed with a through hole 64 for passing a treatment tool such as the high frequency knife 30 therethrough. Operation wires 66 are respectively connected one each to opposite ends 65 on either side of the through hole 64. When one operation wire 66 is pulled and the other operation wire 66 is pushed by operation on the hand side, the main body 63 is rotated around the pin 62 by a predetermined angle. If the pushing/pulling of the operation wires 66 is reversed, the main body 63 is rotated in the reverse direction by a predetermined angle. If the pushing/pulling of the operation wires 66 is continually repeated, the main body 63 is swung.

In this treatment tool operation stage 60, by swinging the treatment tool operation stage 60, the distal end of the treatment tool can be swung in an approximately left-and right direction of an observation image observed by an operator. For example, when a tissue is incised by the high frequency knife 30, the tissue can be incised or the like simply by operating the treatment tool operation stage 60, without bending the insertion portion 3 of the endoscope 51. By adjusting the operation amount of these two operation wires 66, a minute operation can be realized. The detailed construction and the usage of the treatment tool operation stage 60 are disclosed in U.S. Pat. No. 6,824,509. The contents disclosed in U.S. Pat. No. 6,824,509 are incorporated in the present embodiment.

Moreover, the first device may have an ultrasonic diagnostic function or a narrow band imaging (NBI) observation function.

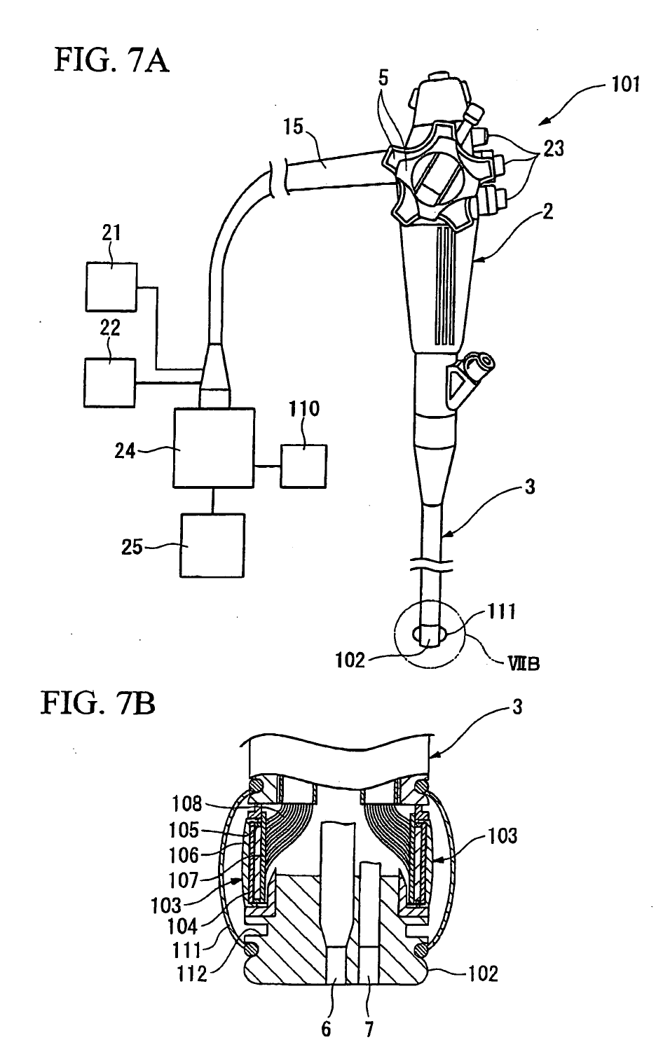

FIG. 7A and FIG. 7B show an example of a device having an ultrasonic diagnostic function. As shown in FIG. 7A and FIG. 7B, an ultrasonic endoscope (ultrasonic observation device) 101 which can be jointly used for ultrasonic diagnosis, has both an endoscope function having the optical observation device 6, and an ultrasonic function capable of observing a tomogram (ultrasonic tomogram) inside a living body. The distal end of the insertion portion 3 is fixed with a distal hard part 102. The distal hard part 102 has an approximate column shape, having a distal face arranged with the observation device 6 and the illuminating device 7 in parallel to the insertion direction, and open ducts (not shown) respectively communicated with the fluid supply channel 8 and the suction channel 9. The outer peripheral surface of the distal hard part 102 is arranged with an ultrasonic transducer 103. The ultrasonic transducer 103 is made from a piezoelectric material or the like, and comprises a plurality of oscillators 104 that are arranged in a torus shape as a whole, an electrode 105, an acoustic lens layer 106, and a predetermined circuit board 107. The construction is such that an ultrasonic wave is oscillated and received in a direction approximately orthogonal to the insertion direction.

The electrode 105 is connected with a signal cable 108 via the circuit board 107. The signal cable 108 is connected to an ultrasonic controller 110 through the inside of the ultrasonic endoscope 101. Furthermore, the distal hard part 102 is attached with a balloon 111. This balloon 111 is expanded by a fluid supplied from a balloon water supply/drainage duct 112 provided on the distal hard part 102. The ultrasonic endoscope is not limited to the forward view radial type as shown in FIG. 7B, and may be for example a convex type or a linear type.

When the ultrasonic endoscope 101 is used, the insertion portion 3 is inserted into the stomach while observing inside the body by the observation device 6 provided on the distal end. Using an ultrasonic wave oscillated by the ultrasonic transducer 103, a tomogram outside of the stomach ST is obtained. By means of the tomogram, it can be confirmed whether or not another organ or the abdominal wall AW is close to the outside of the stomach ST. Moreover, it can be confirmed where the insertion portion 3 of the ultrasonic endoscope 101 is in the stomach ST. By using ultrasonic waves, the condition of the abdominal cavity AC can be confirmed in a non-contact manner, prior to incision of the stomach ST.

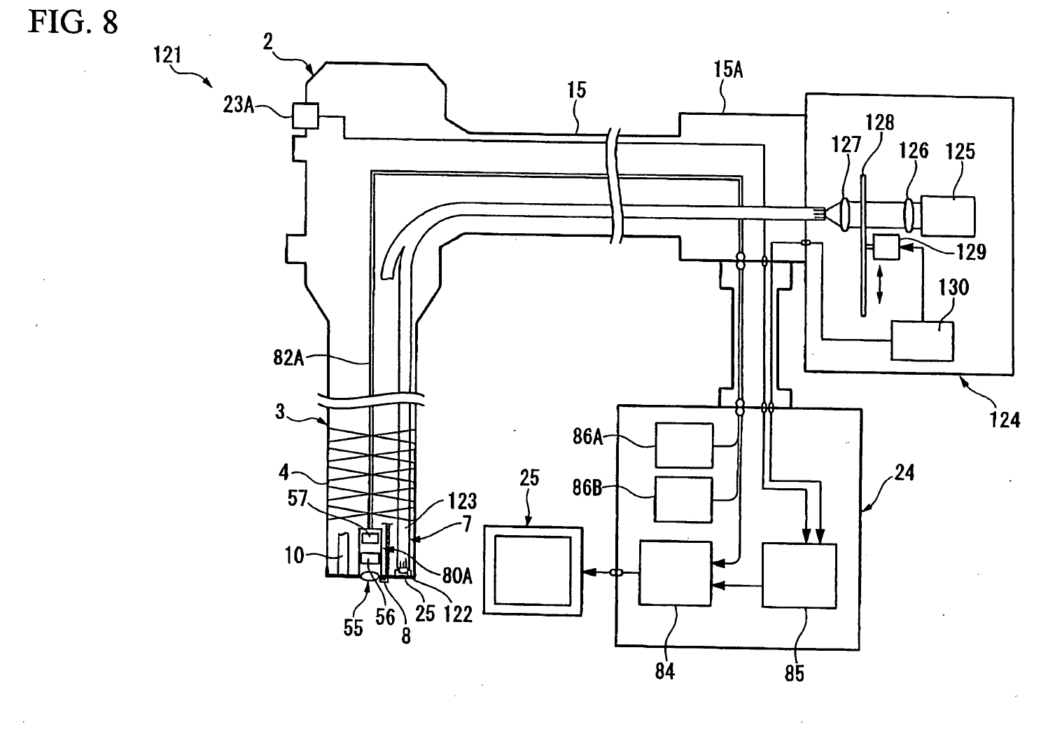

FIG. 8 shows an example of a device having a narrow-band observation function. As shown in FIG. 8, a narrow-band endoscope (narrow-band observation device) 121 in which the narrow-band observation function can be jointly used, has an observation device 80A and an illuminating device 7 on the distal end of the insertion portion 3. The illuminating device 7 has a lens 122 and an optical fiber 123, so as to introduce light from a light source unit 124 on the hand side, by the optical fiber 123. In the present embodiment, in the light source unit 124, a xenon lamp 125 serving as a white light source is used, and a rotating filter 128 for limiting wavelengths of white light is arranged on an optical path of parallel light formed by lenses 126 and 127. In the rotating filter 128, red (R), green (G), and blue (B) are sequentially arranged in the rotation direction. By rotating the rotating filter 128, a beam belonging to a waveband of R, G, and B with a wavelength width in the narrow-band (for example, 20 to 40 nm of full width at half maximum) is incident on the optical fiber 123. A motor 129 which rotates the rotating filter 128 is controlled by the light source controller 130. A synchronization signal, for specifying a position of the rotating filter 128, that is, a waveband of the illuminating light, is output from the light source controller 130 to the controller 85. As a result, the narrow-band endoscope 121 can take in an image synchronously with the waveband of the illuminating light (rotation of the rotating filter 128).

When the narrow-band endoscope 121 is used, blood vessels in the vicinity of a mucosal surface are more emphatically displayed compared to a case where a living tissue is observed by irradiating normal light. Therefore, it can be used as an auxiliary function when diagnosing an affected area. That is, when blue light having a short wavelength is irradiated, an image of blood vessels and the like in the vicinity of a surface layer can be obtained due to an absorption property and a scattering property in the living tissue. When green light having a longer wavelength than that of blue light is irradiated, an image of blood vessels and the like in a deeper area than that of blue light can be obtained. When red light having a long wavelength is irradiated, an image of blood vessels and the like in a much deeper area can be obtained. In this manner, by using the narrow-band endoscope 121, a position of an orifice can be determined so as to avoid thick blood vessels and the like, in a non-contact manner.

Next, insufflation of the abdominal cavity AC is performed. As a method of insufflation of the abdominal cavity AC, a insufflation needle 201 pierced from the abdominal wall AW side is used, or the insufflation needle 201 introduced through a natural orifice is passed through the stomach ST, and is introduced into the abdominal cavity AC. The timing of insufflation is not limited to this, or it may not necessarily be performed. However, if it is performed at this stage, an orifice is formed in the stomach ST after a space is formed between the abdominal wall and a hollow organ to be formed with an orifice (in the present embodiment, between the abdominal wall AW and the stomach ST). Therefore, separate work for forming a space between the abdominal cavity AC and the stomach ST can be omitted. When insufflation is performed after a first medical procedure described later, if the device has a gas supply function, since insufflation can be performed using the gas supply function of the device introduced into the abdominal cavity AC, there is no need for piercing the insufflation needle 201. Furthermore, as a means of keeping a space rather than by insufflation, a publicly known lifting method or the like may be used.

After insufflation of the abdominal cavity AC has been performed, then using a first device 220, an orifice for approaching the abdominal cavity AC is formed in the anterior wall of the stomach ST.

The first device 220 used for forming an orifice in the stomach ST may have a narrow-band observation function facilitating confirmation of the blood vessels in the vicinity of a surface layer as described above, or an ultrasonic diagnostic function (this may be an ultrasonic probe capable of being inserted into the work channel 10 of the endoscope 1) capable of confirming whether or not an organ exists on the opposite side of a site to be formed with an orifice. Furthermore, it may be a device in which a narrow-band observation function, an ultrasonic diagnostic function, and a normal optical observation can be jointly used. If such functions are provided, then when an orifice is formed, observation of an area in the vicinity of an orifice formation position is facilitated. Therefore, the procedure up to formation of the orifice can be more readily performed.

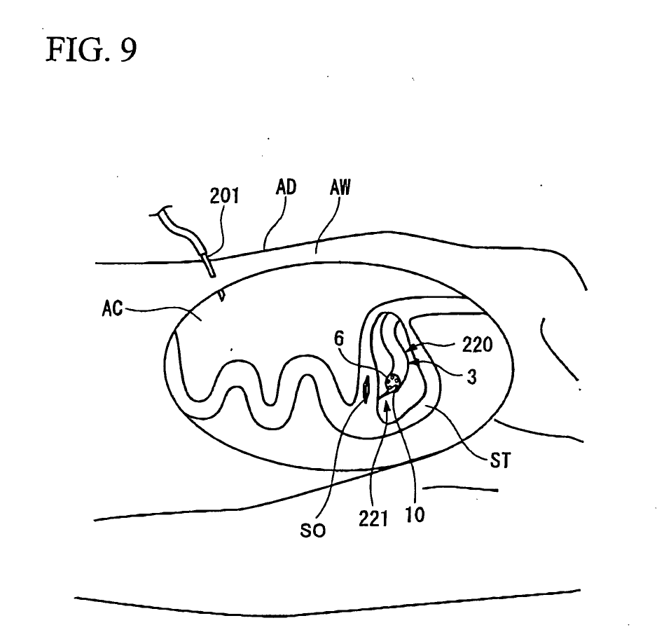

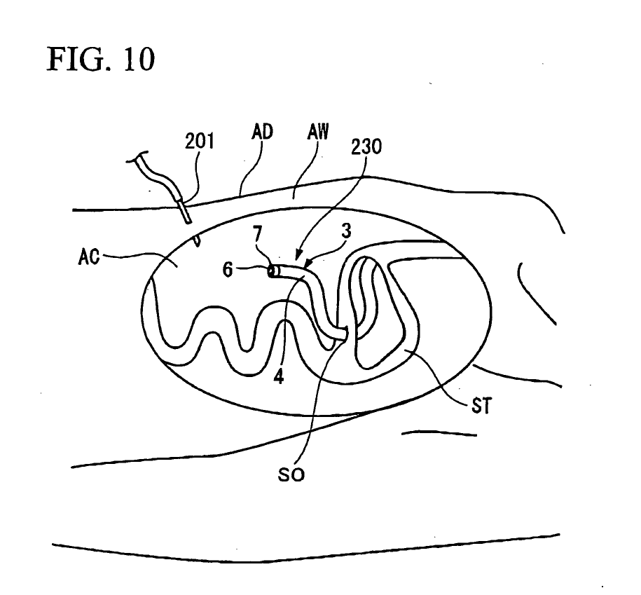

As shown in FIG. 9, if a forward view type endoscope 1 is used as the device 220, since the view field direction and the insertion direction are matched, it can be readily inserted into the body, and incision can be readily performed if there is an incision-planned position ahead of the insertion direction. A treatment tool 221 for incision (for example, a high frequency knife) that has been passed through the work channel 10 is passed through, and the stomach wall is incised along the marking to form an orifice SO.

Moreover, the construction of the lateral view type endoscope 41 (refer to FIG. 3A) is such that the view field center is approximately orthogonal to the insertion direction. If an orifice for approaching the abdominal cavity AC is formed in the stomach ST, the endoscope 41 is preferably provided in the anterior wall site of the stomach ST. Such type of endoscope is used as a first device 220 and is inserted so that the view field direction (in other words, the direction of the view field center) approximately matches the front direction of the patient PT, thereby enabling the anterior wall site of the stomach ST to be more readily specified. Moreover, it is not limited to the anterior wall of the stomach ST, and by inserting the endoscope so that the view field direction approximately matches a direction where the orifice SO is desirably formed, an operation for specifying the site to be formed with the orifice SO can be more readily performed. If an oblique view type endoscope 51 (refer to FIG. 4) is used as the device 220, similarly to the lateral view type endoscope 41 shown FIG. 3A and FIG. 3B, since ahead of the view field center has an angle with respect to the insertion direction, there is an advantage of further facilitating specification of the site to be formed with an orifice, compared to the case where a forward view type endoscope is used. Moreover, in an oblique view type endoscope, since the view field is inclined with respect to the insertion direction, an incision can be readily performed if there is an incision-planned position in such a direction.

Once the orifice SO has been formed in the stomach wall, the device 220 is withdrawn, and instead another device 230 is inserted (device exchange). The replacement device includes a sterilized forward view type endoscope 1, an oblique view type endoscope 51 having the view field center inclined forward with respect to the insertion direction, a multistep bendable endoscope described later, and a treatment endoscope described later.

If it is replaced with the abovementioned forward view type endoscope 1, since the insertion direction and the view field direction are approximately matched, an excellent insertion property (approaching property) in the abdominal cavity AC can be obtained. Moreover, since there is no complicated operation system, then while maintaining excellent operability, an approach to a target site in the abdominal cavity AC, and confirmation and observation of the target site (or ensuring a route to the target site) can be performed. If it is replaced with the oblique view type endoscope, then for example while the abdominal wall AW is captured at the top of the observation image, and furthermore ahead of the insertion direction is confirmed, insertion inside the abdominal cavity AC can be performed. If the lateral view type endoscope 41 is used as the device 220, then by replacing it with the forward view type endoscope 1 or the oblique view type endoscope 51, aspects of operability and insertion properties in the abdominal cavity AC can be further improved. When the oblique view type endoscope 51 is used as the device 220 to form an orifice, and an approach is made to inside the abdominal cavity AC, it may be replaced with a forward view type endoscope having an insertion portion 3 longer than the insertion portion of the device 220 (or visa-versa).

Moreover, if it is replaced with a sterilized multistep bendable endoscope or a treatment endoscope, examples of which are shown below, excellent treatment properties can be obtained, if a device which does not require complicated operations is used until the orifice SO is formed, and a treatment is applied to the target site after an approach is made to the target site inside the abdominal cavity AC. Furthermore, by replacing with a sterilized device, a medical procedure can be performed using a cleaner device inside the abdominal cavity AC.

Next, the device is introduced into the abdominal cavity AC through the formed orifice SO. By operating the device, the distal end is made to approach the target site. An orifice is provided in a hollow organ, and by approaching the inside of the abdominal cavity through this orifice, access to an organ in the abdominal cavity AC, or to the abdominal cavity AC side of a hollow organ, and the like, is possible. FIG. 10 shows a device 230 entering from the orifice SO in the stomach ST into the abdominal cavity AC.

The abovementioned device (first device) 220 may be used until an approach is made to the target site inside the abdominal cavity AC. After confirmation or observation of the target site (or confirmation of a route to the target site) using the device 220, the first device may be withdrawn from the body and replaced with the device 230.

Moreover, the device (second device) 230 may have the abovementioned NBI function or a special optical observation function such as a fluorescent observation described later. Furthermore, the device 230 may have a function of obtaining a self impelling force in the insertion direction, for example by rotating a coiled (spiral) insertion portion 3. In this case, insertion inside the abdominal cavity AC is further facilitated.

Moreover, the insertion portion of the device 230 may have a shape detecting function, an example of which is disclosed in U.S. Pat. No. 6,689,049. The insertion portion is provided with, for example a plurality of magnetic field generating coils which generate a magnetic field, as a shape detecting device, and a magnetic field is generated by electrically charging the magnetic field generating coils. This is detected by an antenna outside the body. A signal based on the detected magnetic field generated by the respective magnetic field generating coils, is A/D converted, and then calculation for position detection or computation for position estimation is performed, so as to obtain estimated positional data with respect to the respective magnetic field generating coils. This positional data is sent to a shape image generation section, and then subjected to graphic processing such as interpolation processing which interpolates intervals from the obtained respective discrete positional data, so as to estimate the shape of the insertion portion of the endoscope, and to form an image corresponding to the estimated shape.

If such a shape detecting device is provided on the insertion portion, it becomes unnecessary to irradiate X rays on a patient for an extended time in order to know the shape of the insertion portion, and hence the burden on the patient can be decreased. The detailed construction and the usage of the shape detecting device are disclosed in U.S. Pat. No. 6,689,049. The contents disclosed in U.S. Pat. No. 6,689,049 are incorporated in the present embodiment.

If the abovementioned forward view type endoscope 1 or oblique view type endoscope 51 is used until an approach is made to the target site in the abdominal cavity AC, then after it reaches the target site and the target site is confirmed (or the target site is observed), it is replaced with a device suitable for treatment when treatment of the target site is performed.

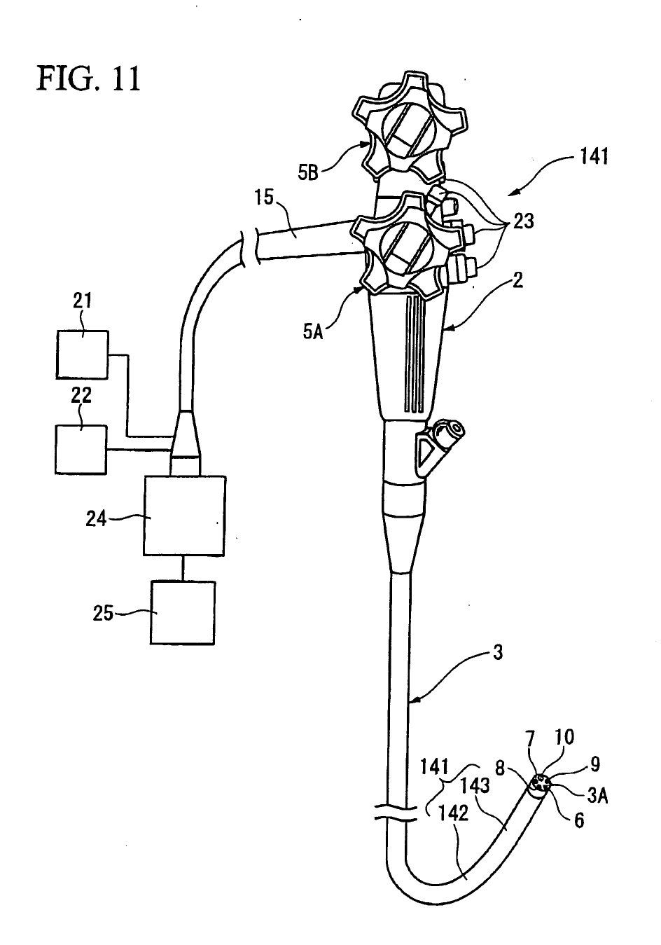

FIG. 11 shows one aspect of a multistep bendable endoscope as an example of a device suitable for treatment. The respective bending sections 142 and 143 have a construction where a plurality of bendable bridges is respectively rotatably connected in the lengthwise direction. The distal end of the first bending section 142 is fixed with a wire (not shown) for bending operation, and this wire is connected to a first angle knob 5A of the operation portion 2. The distal end of the second bending section 143 is fixed with a wire (not shown) for bending operation, and this wire is connected to a second angle knob SB of the operation portion 2. In this endoscope 141, the first and the second bending sections 142 and 143 can be independently curved by providing two operation systems for the second angle knob SB in addition to the first angle knob 5A. The detailed construction and the usage of the multistep bending are disclosed in U.S. Pat. No. 6,899,673. The contents disclosed in U.S. Pat. No. 6,899,673 are incorporated in the present embodiment.

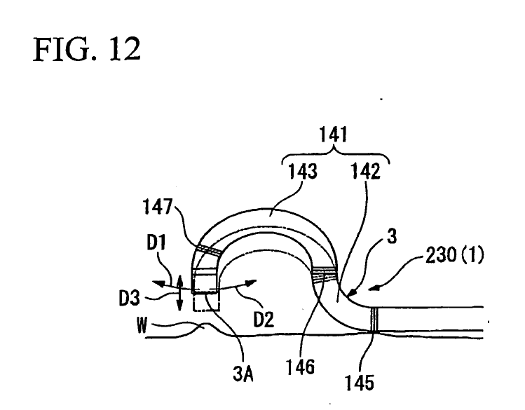

In an endoscope having only one bending section, upon approach to the target site by bending the bending section, the distance between the distal end of the endoscope and the target site may be too close in some cases. On the other hand, if a multistep bendable endoscope comprising a plurality of bending sections (in the embodiment, comprising two bending sections) is used, then as shown in FIG. 12, by bending the first bending section and the second bending section respectively in the opposite directions, an approach of the distal end can be made to the target site without bringing it too close. Therefore the workability when performing observation or treatment can be further improved. Moreover, if the first bending section 142 is curved as shown by the broken lines while the curved condition of the second bending section 143 is fixed, the distal face 3A can be moved in the arrow D3 direction while it is faced to the target site W. As a result, the distance between the distal face 3A and the target site W can be adjusted. Moreover, by suitably operating the two bending sections 142 and 143, the position of the distal face 3A can be minutely adjusted.

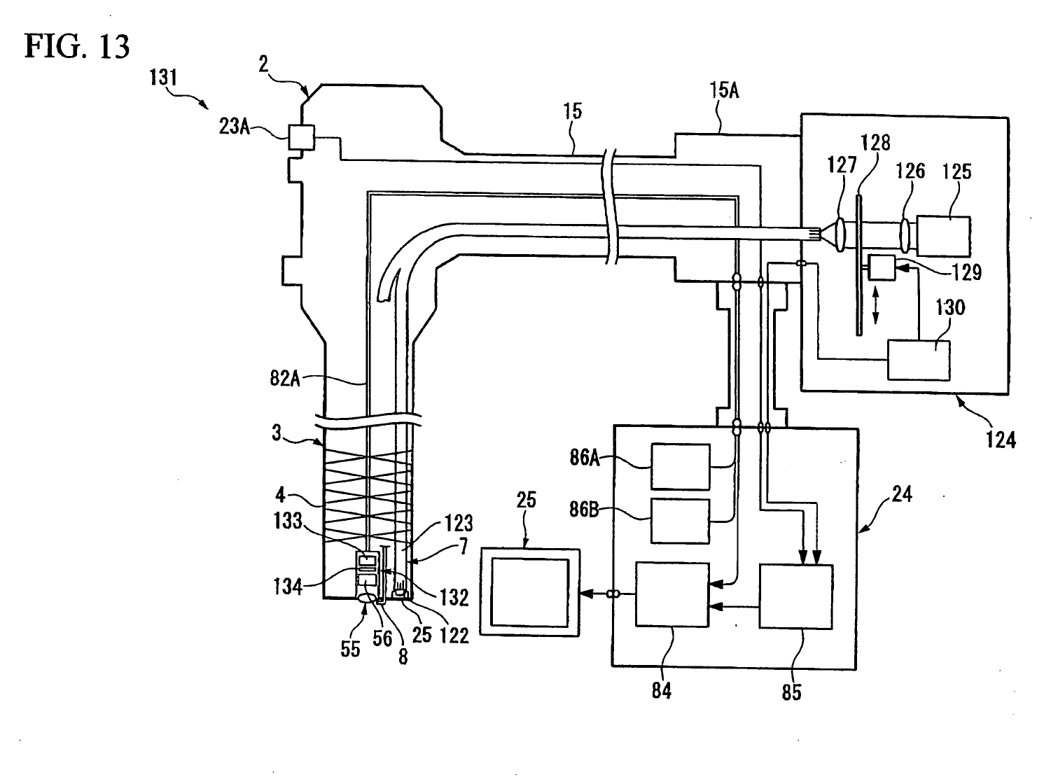

FIG. 13 shows an example of a construction of an endoscope having a fluorescent observation function serving as another form of the special optical observation function. An endoscope 131 irradiates exciting light on a living tissue, and captures fluorescence emitting from the living tissue due to the exciting light, so as to observe it. The distal end of the insertion portion 3 is arranged with an observation device 132 and an illuminating device 7. The observation device 132 has a CCD 133 as an imaging device. A device having a high sensitivity is used for the CCD 133, and the front face of the CCD 133 is arranged with a filter 134 which passes light having wavelengths of 480 nm to 600 nm which correspond to fluorescence wavelengths from the living body.

The illuminating device 7 irradiates a living body with exciting light of a blue component (wavelength of 400 nm to 500 nm) formed by the rotating filter 128. The living body is excited by the exciting light, and thereby emits weak fluorescence, and the fluorescence which has passed through the filter 134 is input to the CCD 133. The detailed construction and the usage of the fluorescent observation endoscope 131 are disclosed in U.S. Pat. No. 6,902,527. The contents disclosed in U.S. Pat. No. 6,902,527 are incorporated in the present embodiment. This fluorescent observation endoscope 131 can be used when performing diagnosis using the phenomena that fluorescence emitting from a normal tissue by irradiation of exciting light thereon is different from that of an affected tissue.

Using the fluorescent observation endoscope 131, by observing fluorescence emitting from a living body by irradiation of exciting light, it can be readily confirmed in a non-contact manner, whether or not an affected site exists. Therefore, it becomes possible to improve the reliability of diagnosis, and to shorten the time required for diagnosis.

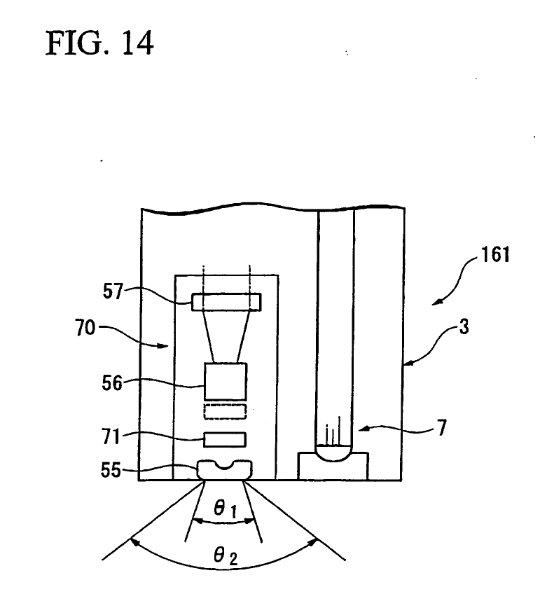

Furthermore, as shown in FIG. 14, the device 230 may comprise an observation device with a zoom function. An observation device 70 with a zoom function has a movable lens 71 which can be moved back-and-forth in the optical axis direction, between an object lens 55 and an imaging lens group 56. If the movable lens 71 is arranged in the position shown by the solid line in FIG. 14, the view field angle becomes θ1. If the movable lens 71 is moved toward the CCD 57 and brought closer to the imaging lens group 56, a wide view field angle θ2 can be obtained. In an endoscope having a fixed view field angle, since the observation view field is fixed in a narrow area ahead of the endoscope, then if the tip of the treatment tool goes even slightly away from the tip of the endoscope, the treatment tool becomes too small to watch, or the treatment tool goes beyond the observation view field. Moreover, when the insertion portion 3 is curved, if the observation view field is narrow, the target position is easily lost. On the other hand, in an endoscope 161 comprising the observation device 70 whose view field angle of the observation view field can be changed, then after widening the observation view field by changing the view field angle, the bending section 4 is curved or a treatment tool is operated, thus facilitating a medical procedure. The observation device 70 may be provided on the oblique view type endoscope 51.

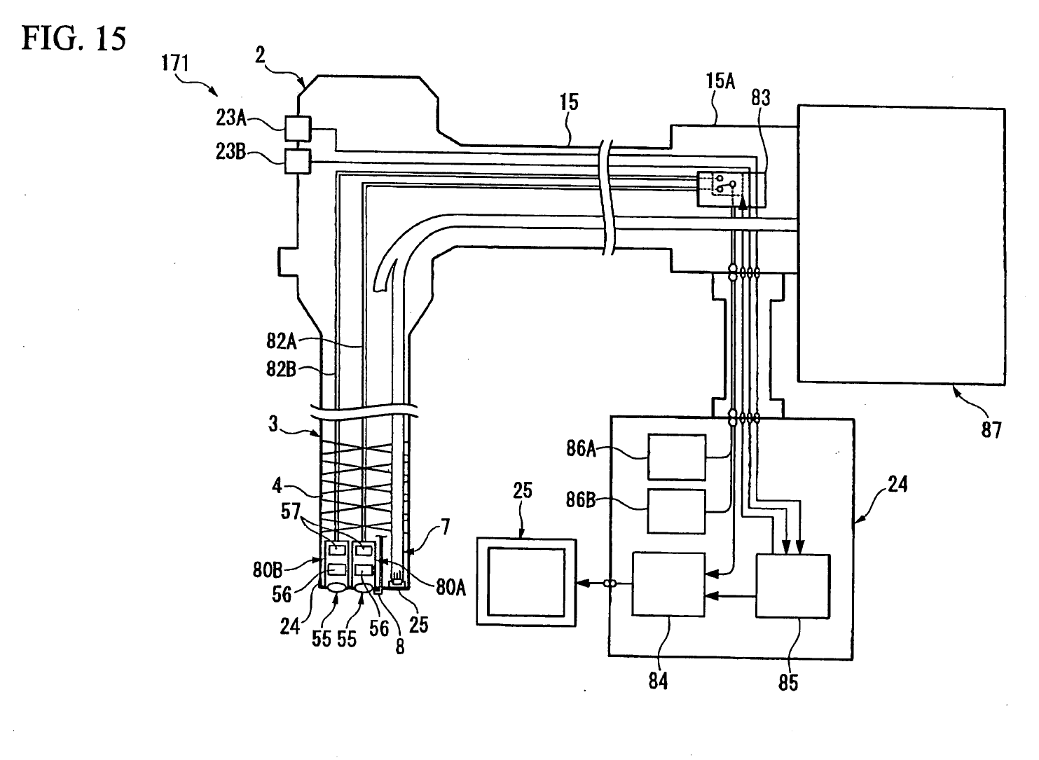

Moreover, the device 230 may comprise a plurality of observation devices. FIG. 15 shows an example of a construction of an endoscope comprising a plurality of observation devices. The distal end of an endoscope 171 is arranged with two observation devices 80A and 80B side by side. The observation devices 80A and 80B have one each of an object lens 55, an imaging lens group 56, and a CCD 57. By changing the arrangement of the imaging lens group 56, the view field angle is changed. The outputs of these two observation devices 80A and 80B are respectively connected with cables 82A and 82B. The respective cables 82A and 82B are led into a connector 15A of a universal cable 15, and connected to a switching circuit 83. The switching circuit 83 switches an output signal from either one of the observation devices 80A and 80B to input into an image processor 84 of the controller 24, and the switching process is performed based on a command from the control circuit 85.

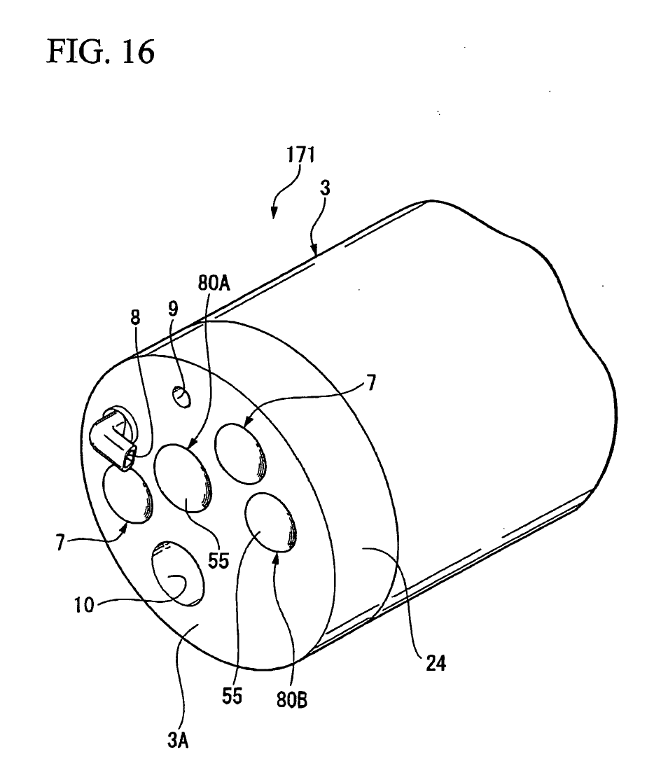

Moreover, the cables 82A and 82B are also connected with driving circuits 86A and 86B for controlling the respective CCDs 57. The control circuit 85 receives an operation of switches 23A and 23B of the operation portion 2 of the endoscope 171, and sends a command to the switching circuit 83. For example, if a switch 23A is pushed, a video image of the observation device 80A is displayed through the image processor 84 on a monitor 25. Moreover, images of two observation devices 80A and 80B may be always displayed in one monitor 25. Instead of changing the arrangement of the imaging lens group 56, a wide-angle lens may be used for the object lens 55 of the observation device 80A, and a lens having a narrower view field angle than that of the wide-angle lens, may be used for the object lens 55 of the observation device 80B, so as to change the view field angles. In addition, different lenses may be used for the imaging lens group 56, or another lens may be inserted. Furthermore, the construction may be such that one observation device is provided with a special optical observation function described later. FIG. 16 shows the distal end of the insertion portion.

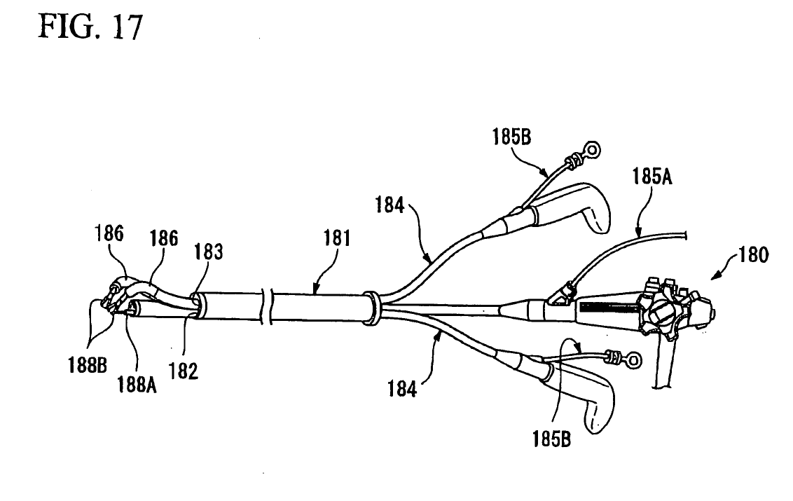

FIG. 17 shows an example of the abovementioned treatment endoscope, showing an example of another form of the endoscope suitable for treatment. The treatment endoscope shown in this example is characterized in that approach of treatment members (or distal ends of arms for treating the target site) can be made from different directions other than ahead of the view field center of the endoscope, to the target site. An endoscope system 180 has an overtube 181 through which the endoscope 1 is passed. The overtube 181 is provided with a channel 182 for passing the endoscope 1 therethrough, and two treatment tool guide insertion tool channels 183 comprising channels in which the distal end of the treatment tool is rotatable, and the treatment tool is insertable and/or withdrawable. Into the treatment tool guide insertion tool channels 183, treatment tool guide insertion tools 184 (arms for treating the target site; also called treatment arms) are inserted one by one in a back-and-forth movable manner and in a rotatable manner. The treatment tool guide insertion tool 184 shown in FIG. 17 is provided with a bending section 186 having approximately the same construction as that of an insertion portion of a publicly known endoscope, that is, a tubular insertion portion having a metal spiral tube covered with a metal netlike braid, and is constructed to perform a bending operation according to the operation of an operation tube, in the distal area. Similarly to a bending section of a publicly known endoscope, this bending section 186 is constructed to be bendable by pivoting a pair of adjacent bendable bridges arranged side by side in the axial direction of the insertion portion, and by moving back-and forth a bending operation wire fixed to the distal area of the bending section. Moreover, by this bending section 186, the distal end is curved toward the central axis of the insertion portion of the endoscope. Therefore, the treatment endoscope (a device for treatment in the abdominal cavity) is characterized in the point that the construction is such that an approach of the distal end of a treatment tool for treatment from a different direction other than ahead of the view field center of the observation device (imaging device of the endoscope in the aspect shown in FIG. 17), can be made with respect to the target site. The shape of the bending section 186 can be operated by a bending operation wire (not shown) passed through the treatment tool guide insertion tool 184, on the hand side. The diameter of the treatment tool guide insertion tool 184 is small, and the outer diameter of the overtube 181 is about a size capable of passing through a hollow organ of a patient.

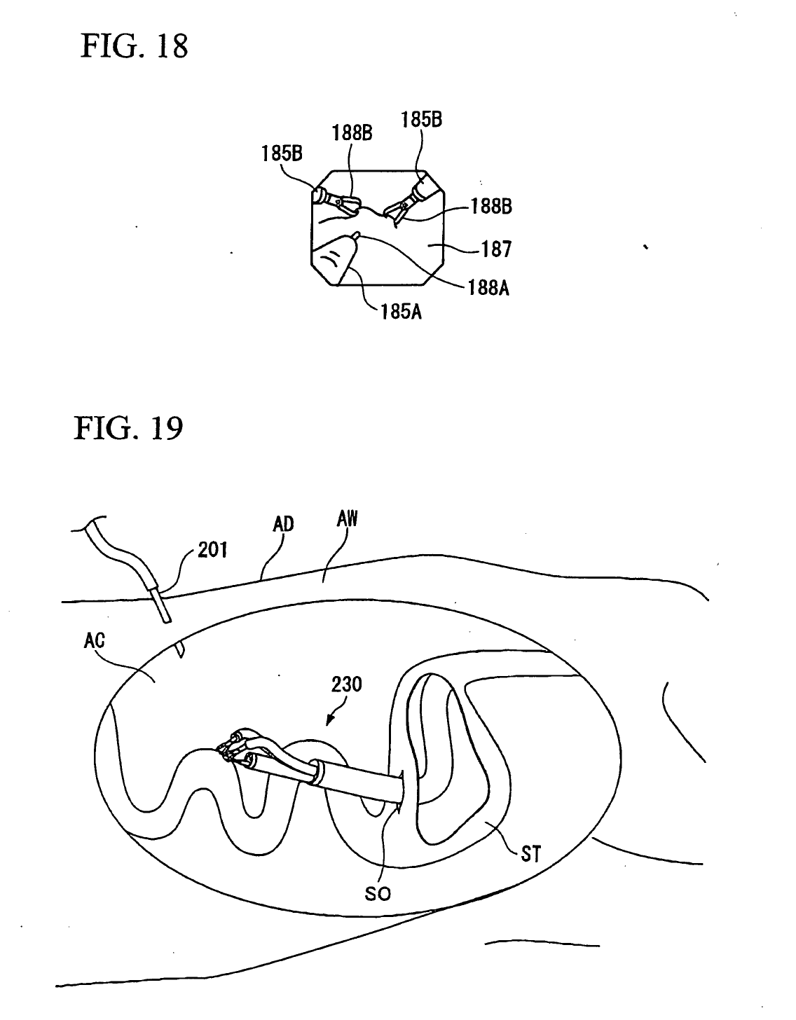

If such an endoscope system 180 is used as a second device, a plurality of treatment tools 185A and 185B can be simultaneously used when performing treatment on the target site. Moreover, in the aspect shown in FIG. 17, for the insertion portion of the treatment tool guide insertion tool 184, there is employed a similar construction to that of an insertion portion of a publicly known endoscope, that is, a bending section provided on a flexible distal area, and formed from a metal spiral tube covered with a netlike braid. Therefore the rigidity of the insertion portion can be improved. As a result, a treatment (as an example, grasping, resection, and abrasion of a tissue) can be performed on a tissue of the target site (for example, an organ), with a greater force compared to a normal flexible treatment tool, and hence the treatment properties are further improved. By providing a plurality of work channels 10 in the endoscope, a plurality of treatment tools can be simultaneously used. However, since intervals between the work channels 10 become narrow, movable ranges of the treatment tools 185A and 185B become narrow. By passing the treatment tool 185B through the treatment tool guide insertion tool 184, the movable area of the treatment tool 185B can be broadened, facilitating the medical procedure. Moreover, the view field direction of the observation device 6 (refer to FIG. 2A), and the direction of approach of the treatment tool 185B to the target site can be made significantly different. For example, as with the video image 187 of the observation device 6 shown in FIG. 18, the target site and the treatment portions 188A and 188B on the distal ends of the respective treatment tools 185A and 185B can be respectively observed. Consequently, the view field required for a medical procedure can be readily ensured. The number of treatment tool guide insertion tools 184 is not limited to two. Moreover, the bending section 186 of each treatment tool guide insertion tool 184 is not limited to one.

Next, as shown in FIG. 19, using a device 220, a desired treatment is performed on the target site in the abdominal cavity AC. The treatment mentioned here corresponds to procedures such as observation, biopsy, incision, anastomosis, and extirpation of an organ. For example, medical procedures such as observation of an organ in the abdominal cavity, biopsy of the liver or pancreas, cautery of the uterus myoma or liver (and other organs), sterilization treatment, extirpation of the appendix or gallbladder, and operations for obesity, are performed.

In the case where a treatment is performed on the target site, if as a second device, the abovementioned endoscope comprising a multistep bending function, or a treatment endoscope is used, treatment is improved from the viewpoint of treatment properties. Therefore it is effective for performing treatments having a high therapeutic factor. Meanwhile, by using a device which does not require a complicated operation up until an orifice is formed in the wall of the hollow organ (or until confirmation or observation of a target site or a route to the target site), and for which it is easy to specify a site to be formed with an orifice, a procedure can be performed more efficiently. Consequently, overall, shortening of the time required for a medical procedure can be expected.

After the second medical procedure is completed, inside of the abdominal cavity AC is washed. Washing is performed by sending a liquid such as physiological salt solution, from the distal end of the insertion portion to the target site (and its surroundings), using a fluid supply channel, or a treatment tool insertion channel, or the like of the endoscope. The liquid after washing is sucked using the suction channel, or the treatment tool insertion channel.

After washing of the abdominal cavity AC is completed, the device that has been introduced in the abdominal cavity AC is brought back into the stomach ST (hollow organ). Since the stomach wall is left with the formed orifice SO, the orifice SO is closed. That is, communication between the inside of the hollow organ, and the abdominal cavity is blocked.

In the operation of closing the orifice SO, the device that has been brought back into the stomach ST may be used. If suture of the orifice SO is required, a suturing treatment tool is used.

After the orifice SO in the stomach wall is closed, it is confirmed whether or not the orifice SO is closed in a desired condition. The confirmation operation includes visual observation by the observation device 6 of the endoscope 1, and a leak test in which water or air is supplied into the stomach ST or the abdominal cavity AC, to confirm whether or not the air or liquid is leaked from the sutured part. After confirming that the orifice SO is closed in a desired condition, the device is withdrawn, and the series of medical procedures is completed.

The present embodiment is designed such that, upon a medical procedure inside the abdominal cavity AC through the endoscope from the patient PT's mouth, for each step of the medical procedure, the medical procedure is performed by replacing with a device suitable for the step. Upon a medical procedure inside the abdominal cavity, in a method of inserting a device from a natural orifice, and inserting a device from an orifice formed in the hollow organ into the abdominal cavity, devices used at respective steps of the medical procedure are not necessarily the same. Consequently, as in the present embodiment, by replacing with a suitable device for each step of the medical procedure, the medical procedure can be readily and reliably performed. As a result, the load on the patient is reduced.

Up until an orifice is formed in a wall of a hollow organ (or the target site or a route to the target site is confirmed or observed), by using a conventionally and widely used device not requiring complicated operations, or a device which facilitates specifying a site to be formed with an orifice, procedures such as forming an orifice, or confirmation or observation of a route to the target site, can be more efficiently performed. Moreover, when a treatment is performed on the target site, if the abovementioned endoscope comprising a multistep bending function, or a treatment endoscope, is used as a second device, the treatment properties are improved. Therefore it becomes more effective for performing a treatment having a high therapeutic factor. Consequently, overall, shortening of the time required for a medical procedure can be expected.

When the endoscope is replaced, it may be inserted from a different natural orifice. For example, while an endoscope is inserted from the patient PT's mouth, the next endoscope may be inserted from the patient PT's anus, and introduced from an orifice formed in the colon into the abdominal cavity.

### Second Embodiment

The present embodiment is characterized in jointly using an overtube (also called a guide tube) as a flexible guide member.

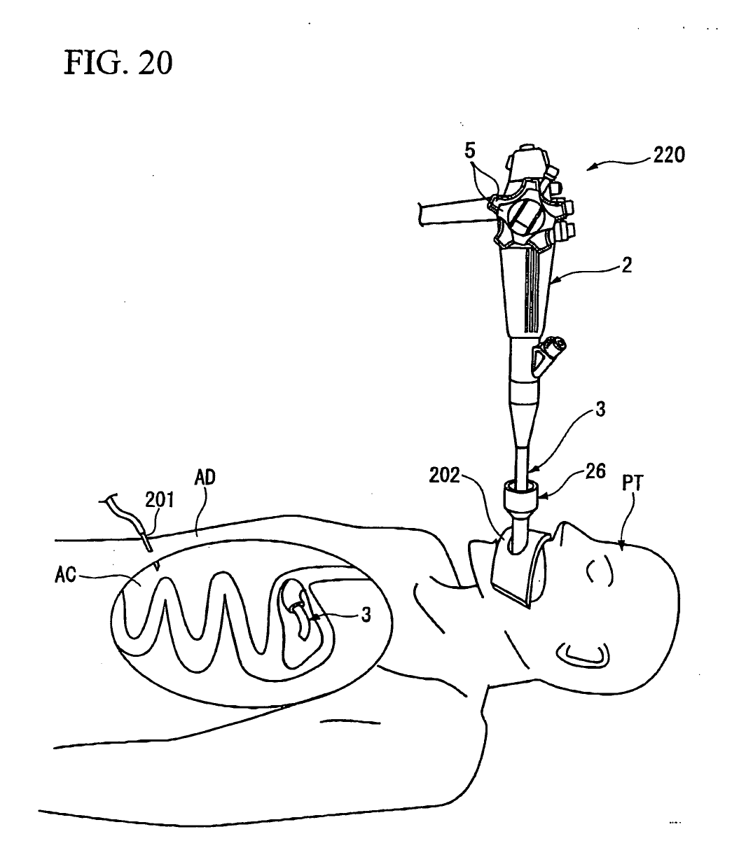

As shown in FIG. 20, when a medical procedure is performed, a device (first device) 220 having the insertion portion, is introduced through a natural orifice into a hollow organ. At this time, the overtube is also inserted through the natural orifice along the insertion portion of the first device 220. As the first device, a similar device to that of the first embodiment may be used.

An overtube 26 has a construction where a slender flexible tube portion 152 is extended from the proximal end 151. The overtube 26 having a size of an outer diameter capable of being inserted from a patient's natural orifice into the body, and an inner diameter capable of passing a device (for example, endoscope 1) through in a back-and-forth movable manner, is used.

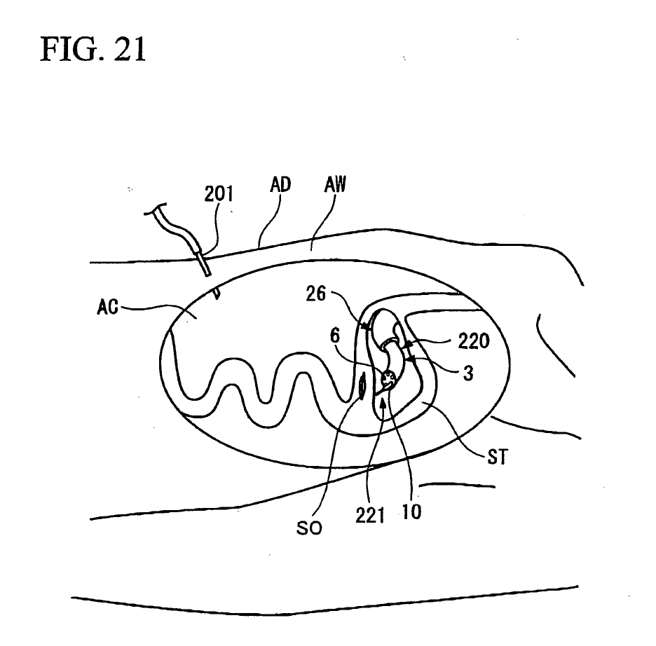

Similarly to the above, after the abdominal cavity AC is insufflated, as shown in FIG. 21, the first device 220 is used, and an orifice for approaching to the abdominal cavity AC is formed, in the anterior wall of the stomach ST, so that an approach can be made to the abdominal cavity AC. Here, except for forming the orifice using a device inserted through the overtube, the embodiment is the same as the first embodiment.

Similarly to the first embodiment, when an orifice SO is formed, if the lateral view type endoscope 41 is used in order to facilitate specifying a site to be formed with an orifice (in the present embodiment, the anterior wall site of the stomach), then at the time when the orifice site is specified (or after the orifice is formed), it may be replaced with another device. If the device is replaced at this stage using the overtube 161, it becomes possible to use an unused device (unused condition where a treatment is not performed in the hollow organ) as a device for approaching the abdominal cavity AC. As the device to be replaced, there is used a sterilized forward view type endoscope 1, or an oblique view type endoscope 51 having the view field center inclined forward with respect to the insertion direction. If it is replaced with the abovementioned forward view type endoscope 1, since the insertion direction and the view field direction are approximately matched, excellent insertion properties (approach properties) in the abdominal cavity AC can be obtained. Moreover, since there is no complicated operation system, then while maintaining excellent operability, after an approach is made to a target site in the abdominal cavity AC, confirmation and observation of the target site can be performed. If it is replaced with the oblique view type endoscope 51, then for example while the abdominal wall AW is captured at the top of the observation image, and furthermore ahead of the insertion direction is confirmed, insertion inside the abdominal cavity AC can be performed. When the oblique view type endoscope 51 is used as the device 220 to form an orifice, and an approach is made to the abdominal cavity AC, it may be replaced with a forward view type endoscope having an insertion portion longer than the insertion portion of the device 220 (or visa-versa). The overtube used in the second embodiment desirably has a shape retention (shape fixation) function. Shape retention means a capability of retaining the shape after the overtube is deformed into a predetermined shape.

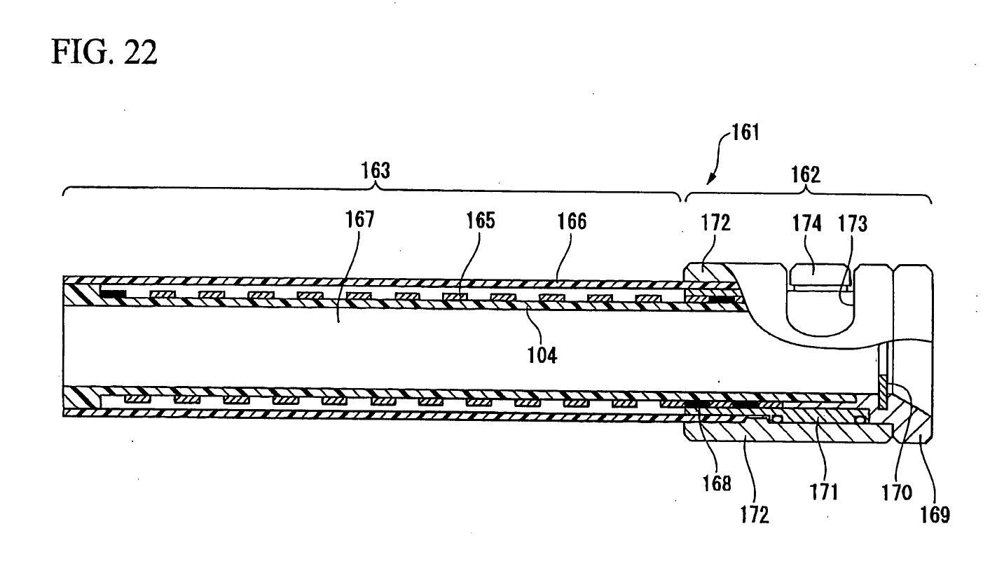

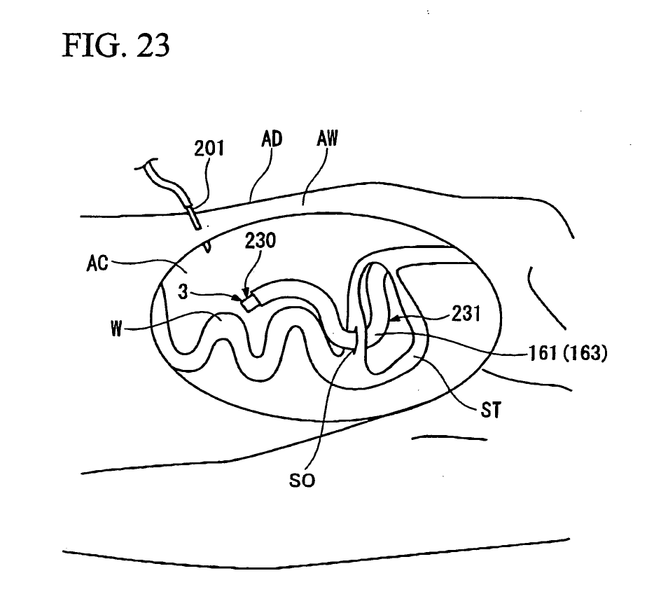

As shown in FIG. 22 as an example, in an overtube 161, a flexible tube portion 163 extended from a proximal operation portion 162 has a cylindrical inner sheath 164. On the outside of the inner sheath 164 is arranged a friction member 165. On the outer circumference of the friction member 165 is further arranged a cylindrical outer sheath 166, constructed such that the friction member 165 is interposed between the outer sheath 166 and the inner sheath 164. The inner sheath 164 is formed from, for example a soft resin, having a through hole 167 for passing an insertion portion of a device such as an endoscope. The outer sheath 166 is formed from, for example a soft resin, and the inner circumferential surface thereof becomes a friction surface which generates a frictional force when it comes into contact with the outer circumferential side of the friction member 165.

The friction member 165 is formed in an approximate tube shape by winding a slender plate member in a spiral shape. The outer circumferential surface of the friction member 165 becomes a friction surface which generates a frictional force when it comes into contact with the inner circumferential surface of the outer sheath 166. The friction member 165 is fixed to the inner circumferential side of the inner sheath 164 on the distal side in the insertion direction, at a fixation portion 168.

The proximal operation portion 162 has an inner sheath fixation member 169. The inner sheath fixation member 169 is fixed with the end of the inner sheath 164 on the hand side. To the inner circumferential side of the inner sheath fixation member 169 is adhered an airtight valve 170 in an inner circumferential flange shape, which forms an airtight structure between a device and the overtube 161, when the device is inserted into the through hole 167. The outer surface of the inner sheath 164 is arranged with a cylindrical transmission member 171. To the transmission member 171 is fixed the friction member 165. Furthermore, the outer sheath 166 is fixed to an outer sheath fixation member 172 so as to cover the transmission member 171. The outer sheath fixation member 172 is fixed with the end of the outer sheath 166, and is formed with a movement groove 173 slenderly in the circumferential direction. In the movement groove 173 is inserted an operation lever 174 in a circumferentially movable manner. The operation lever 174 is fixed to the transmission member 171, and the transmission member 171 can be rotated in the circumferential direction by the operation lever 174. The operation lever 174 may be constructed in an axially movable manner, in addition to the circumferentially movable manner.

In this case, in the overtube 161, the flexible tube portion 163 is passively curved according to the shape of an insertion portion of a device such as an endoscope. At this time, if the operation lever 174 is rotated in a direction to release the winding of the friction member 165, the wound and approximately tubular friction member 165 is radially expanded. Then, the outer circumferential side of the friction member 165, and the inner circumferential surface of the outer sheath 166 come into surface contact, generating a frictional force. In this condition, even if an attempt is made to change the shape of the overtube 161, due to the procedure of frictional force, the curved shape of the flexible tube portion 163 is fixed.

Moreover, in order to bring the fixed flexible tube portion 163 back into the condition capable of changing the shape, the operation lever 174 may be rotated in a direction to tighten the winding of the friction member 165. As a result, the friction member 165 is radially contracted, and the surface contact between the friction member 165 and the outer sheath 166 is released, so that the shape of the flexible tube portion 163 can be changed.

The shape retention function of the overtube is not limited to this. For example, the construction may be such that a plurality of components having holes (lumens in the overtube) for inserting a device into the inside, are arranged so as to define the axial direction of the overtube, and these plurality of components are linked by a wire. In the case of such a construction, when no tension is applied to the wire, it is passively bendable, and if a tension is applied to the wire, adjacent components are mutually engaged and the curved shape can be retained. In this case, the plurality of components may be formed from a plurality of rings having holes which become lumens of the overtube.

Through the orifice SO formed in the stomach ST, the endoscope and the overtube 131 are inserted into the abdominal cavity AC, and the device is operated to make the distal end approach the target site. Using the device as a guide, the overtube 131 is passively curved according to the curved shape of the insertion portion of the device, and the distal end of the overtube 131 is made to approach the vicinity of the target site.

After the distal end of the overtube 131 reaches the vicinity of the target site, the shape retention (shape fixation) function of the overtube 131 is activated so as to maintain the curved shape of the overtube 131.

Then, the device 220 is withdrawn, and another device (second device 230) is inserted using the overtube 131 as a guide, so as to make the second device 230 approach the target site. Even if the device is withdrawn, since the curved shape of the overtube 161 is retained, the distal end of the endoscope can be arranged in the original position merely by exchanging the endoscope. The device 220 may be made to approach the target site, and observation and confirmation of the target site may be performed using the device 220, after which the device 220 is withdrawn and another device (second device) is inserted. As the second device 230, the multistep bendable endoscope and the treatment endoscope shown as an example in FIG. 11 and FIG. 17 are used. These devices have a more complicated construction compared to the above forward view type endoscope 1 and the oblique view type endoscope 51 (for example, in the case of the multistep bendable endoscope, a first operation system for the bending operation of the first bending section, and a second operation system for the bending operation of the second bending section are provided, and in the case of the treatment tool endoscope where the operation is slightly complicated, and the operation portion is large, an operation system for operating the treatment arms is provided). From the viewpoint of the approach property, the forward view type endoscope 1 and the oblique view type endoscope 51 are superior. Using such a forward view type endoscope or oblique view type endoscope as a device for approaching to the target site, the overtube is introduced to the vicinity of the target site, and the device is replaced with a device suitable for treatment, so that the approach property to the target site can be further improved. Other features are similar to the first embodiment.

After washing of the abdominal cavity AC is completed, the endoscope 220 serving as the second device, and the overtube 161 are brought back into the stomach ST (hollow organ).

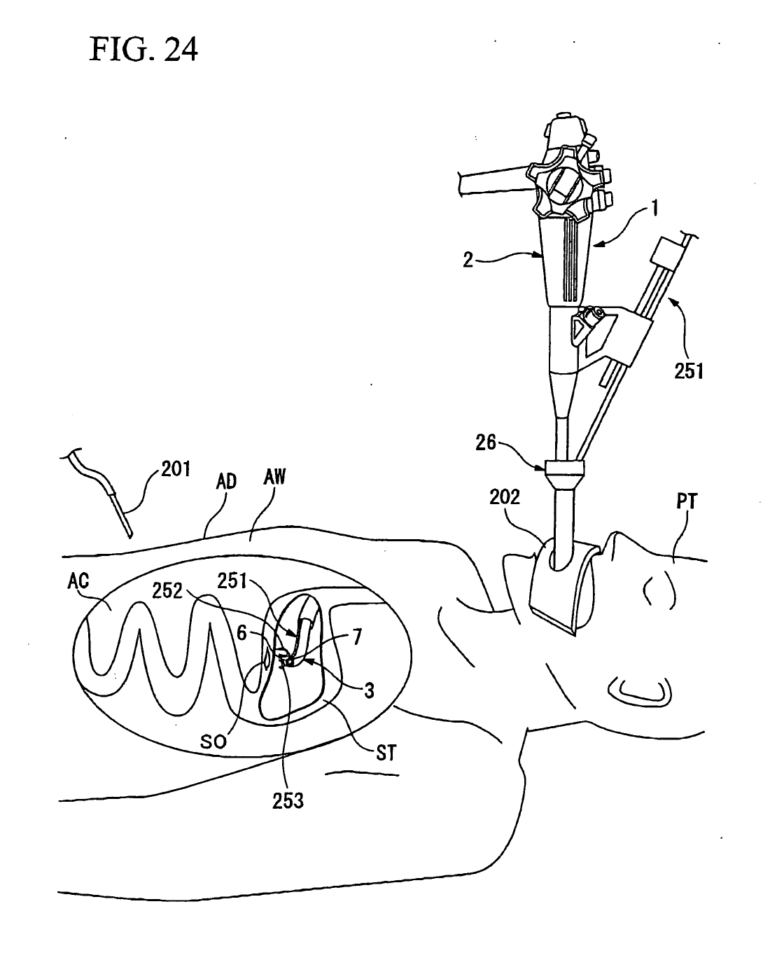

Since the stomach wall is left with the formed orifice SO, the orifice SO is closed by suturing or the like (communication between the inside of the hollow organ, and the abdominal cavity is blocked). In the operation of closing the orifice SO, the device that has been brought back into the stomach ST may be used. If suture of the orifice SO is required, a suturing treatment tool is used. FIG. 24 shows an example of the suturing treatment tool. In this example, a suture device 251 is arranged along the outside of the endoscope 1. The construction is such that the distal end of the suture device 251 is provided with a pair of openable/closable forceps members 252 and 253 attached with a needle and a thread, so that the stomach wall can be sutured. The endoscope for washing may be used as is, for the suturing. The suture device 251 may be another suturing treatment tool.

After the orifice SO in the stomach wall is closed, it is confirmed whether or not the orifice SO is closed in a desired condition. Similarly to the above, the confirmation operation includes visual observation of an endoscopic image, and confirmation of the presence/absence of fluid leakage. After confirming that the orifice SO is closed in a desired condition, the endoscope 1 and the overtube 161 are withdrawn, and the series of medical procedures is completed.

In the present invention, if the overtube 161 capable of shape retention (fixation) is used, then after the distal end of the overtube 161 is guided to the target site in the abdominal cavity, the shape of the overtube 161 can be retained. Therefore, the device after replacement can be more readily guided to the target site, and the time required for a medical procedure can be shortened. Other effects are similar to those in the first embodiment.

As mentioned above, while preferred embodiments of the present invention have been described, the present invention is not limited to these. Additions, omissions, substitutions of the construction, and other modifications can be made without departing from the scope of the present invention. The present invention is not to be considered as being limited by the foregoing description, and is only limited by the scope of the appended claims.

The hollow organ formed with the orifice SO is not limited to the stomach ST. For example, it may be an esophagus, a duodenum, a small intestine, a colon, a uterus, a bladder, and the like.

The construction may be such that an output signal from the imager is transmitted through a signal wire passed through the insertion portion, to external equipment such as a signal processor. Moreover, the construction may be such that an output signal from the imager is transmitted to external equipment by wireless means. Furthermore, the illuminating device may be constructed using a publicly known light emission element (for example, a light emitting diode) or the like.

What is claimed is:

1. A medical procedure through a natural body orifice comprising:
introducing a first device having an insertion portion, from a patient's natural orifice into a hollow organ;
performing a first medical procedure using said first device;
withdrawing said first device from the body, and introducing a second device having an insertion portion, from a patient's natural orifice into the body; and
performing a second medical procedure using said second device,
wherein said first medical procedure is performed prior to said second medical procedure for performing said second medical procedure.

2. The medical procedure through a natural body orifice according to claim 1, wherein said performing a first medical procedure comprises forming an orifice for accessing to the abdominal cavity, in a wall of a hollow organ.

3. The medical procedure through a natural body orifice according to claim 2, wherein withdrawing said first device from the body, and introducing a second device into the body comprises withdrawing said first device after forming an orifice for accessing to said abdominal cavity, in the wall of the hollow organ, and then accessing to the abdominal cavity using said second device.

4. The medical procedure through a natural body orifice according to claim 2, wherein withdrawing said first device from the body, and introducing a second device into the body comprises withdrawing said first device after forming an orifice for accessing to said abdominal cavity in the wall of the hollow organ, and confirming a target site existing in the abdominal cavity by using said first device, and then introducing said second device to perform a medical procedure on the target site.

5. The medical procedure through a natural body orifice according to claim 2, wherein forming an orifice comprises forming an orifice in the anterior wall of the stomach.

6. The medical procedure through a natural body orifice according to claim 5, wherein said forming an orifice in the anterior wall of the stomach comprises introducing a lateral view type endoscope as said first device into the stomach, and confirming the anterior wall of the stomach using said lateral view type endoscope.

7. The medical procedure through a natural body orifice according to claim 5, wherein forming an orifice in the anterior wall of the stomach comprises introducing an oblique view type endoscope as said first device into the stomach, and confirming the anterior wall of the stomach using said oblique view type endoscope.

8. The medical procedure through a natural body orifice according to claim 5, wherein said introducing a second device into the body comprises introducing a different type of device from said first device.

9. The medical procedure through a natural body orifice according to claim 5, wherein said introducing a second device into the body comprises introducing a device constructed so that approach of a treatment portion for performing a medical procedure on a target site, can be made from a different direction other than ahead of a view field center.

10. The medical procedure through a natural body orifice according to claim 5, wherein said performing a second medical procedure comprises performing a desired medical procedure on a target site existing in an abdominal cavity.

11. The medical procedure through a natural body orifice according to claim 10, wherein said introducing a second device into the body comprises introducing said second device into the body through a natural orifice through which said first device had been introduced.

12. A medical procedure through a natural body orifice comprising:
introducing a first device having an insertion portion, from a patient's natural orifice into the body;
introducing a flexible guide member from said patient's natural orifice into the body;
performing a first medical procedure using said first device;
activating a shape retention function of said guide member from outside of the body, so as to fix a curved shape of said guide member according to a curved shape of the insertion portion of said first device;
withdrawing said first device from the body,
introducing a second device having an insertion portion, from a patient's natural orifice into the body, along the fixed shape of said guide member; and
performing a second medical procedure using said second device,
wherein said first medical procedure is performed prior to said second medical procedure for performing said second medical procedure.

13. The medical procedure through a natural body orifice according to claim 12, wherein said performing a first medical procedure comprises forming an orifice for accessing to the abdominal cavity, in a wall of a hollow organ.

14. The medical procedure through a natural body orifice according to claim 13, wherein said performing a first medical procedure further comprises using said first device to introduce a distal end of said guide member to a desired site inside an abdominal cavity.

15. The medical procedure through a natural body orifice according to claim 13, wherein forming an orifice comprises forming an orifice in the anterior wall of the stomach.

16. The medical procedure through a natural body orifice according to claim 15, wherein forming an orifice in the anterior wall of the stomach comprises introducing a lateral view type endoscope as said first device into the stomach, and confirming the anterior wall of the stomach using said lateral view type endoscope.

17. The medical procedure through a natural body orifice according to claim 16, wherein forming an orifice in the anterior wall of the stomach comprises introducing an oblique view type endoscope as said first device into the stomach, and confirming the anterior wall of the stomach using said oblique view type endoscope.

18. The medical procedure through a natural body orifice according to claim 15, wherein said introducing a second device into the body comprises introducing a different type of device from said first device.

19. The medical procedure through a natural body orifice according to claim 18, wherein said introducing a second device into the body comprises introducing a device constructed so that approach of a treatment portion for performing a medical procedure on a target site, can be made from a different direction other than the ahead of a view field center.

20. The medical procedure through a natural body orifice according to claim 7, wherein said performing a second medical procedure comprises performing a desired medical procedure on a target site existing in an abdominal cavity.
